(19) United States
(12) Patent Application Publication
(10) Pub. No.: US 2007/0179121 A1
(43) Pub. Date: Aug. 2, 2007

(54) Method of treating pediatric patients with corticosteroids

(76) Inventor: R. Todd Plott, Scottsdale, AZ (US)

Correspondence Address: William J. McNichol, Jr., Esquire;Reed Smith LLP, Intellectual Property, P.O. Box 7990, Philadelphia, PA, 19101, US

(21) Appl. No.: 11/347,708

(22) Filed: Feb. 2, 2006

(51) Int. Cl. A61K 31/573

(52) U.S. Cl. 514/179

(57) ABSTRACT

The present invention comprises a method of treating pediatric patients suffering from an inflammatory or pruritic skin disorder comprising topically applying fluocinonide to an affected area. Treatment according to this method does not result in clinically significant suppression of the HPA-axis.

### FIELD OF THE INVENTION

Clinically, topical corticosteroids are useful for their anti-inflammatory, and anti-pruritic actions. Corticosteroids (or corticoids) are any steroids (lipids that contain a hydrogenated cyclopentoperhydrophenanthrene ring system) elaborated by the adrenal cortex (except sex hormones of adrenal origin) in response to the release of adrenocorticotrophin or adrenocorticotropic hormone by the pituitary gland, or to any synthetic equivalent, or to angiotensin II.

The potency of topical steroid preparations is strongly correlated to their absorption through the skin and activity of the compound. Treatment of the skin prior to application of the topical steroid may also affect the absorption of the compounds into the skin. Treatments with keratolytics or with fat solvents (such as acetone) disrupt the epidermal barrier and increase penetration. Hydrating the skin has also been shown to increase the penetration of the corticosteroids.

Once absorbed through the skin, topical corticosteroids are handled through pharmacokinetic pathways similar to systemically administered corticosteroids. The potencies of corticosteroids vary greatly.

The clinical effectiveness of corticoids is related to four basic properties: vasoconstriction, antiproliferative effects, immunosuppression, and anti-inflammatory effects. Topical steroids cause the capillaries in the superficial dermis to constrict, thus reducing erythema. The ability of a given glucocorticoid agent to cause vasoconstriction usually correlates with its clinical potency. Vasoconstrictor assays are used in the art and by the U.S. Food and Drug Administration for determining the potency of topical corticosteroid preparations. Topical glucocorticoid preparations have been divided in the field into seven classes based on potency based on double-blind clinical studies and vasoconstrictor assays. Class 1 includes the most potent, while class 7 contains the least potent.

Several factors such as the vehicle, the integrity of the epidermal barrier, and the use of occlusive dressings affect the percutaneous absorption and resulting potency of corticosteroids regardless of the intrinsic potency of the glucocorticosteroid (or glucocorticoid) molecule. Further, inflammation and/or other disease processes in the skin increase percutaneous absorption.

The vehicle in which the corticoid is incorporated affects the amount of corticoid that is released in any given period of time and its absorption. The solubility of the corticoid in the vehicle also affects penetration into the skin. Very occlusive vehicles, such as ointments (water-insoluble mixtures of oil and petrolatum), increase the corticosteroid effect because they provide increased hydration of the stratum comeum and increase the skin's permeability. By covering the skin with an occlusive dressing such as plastic wrap, this effect can be heightened as much as 100-fold. The solubility of the corticoid in the vehicle also affects penetration into the skin.

Creams, which are suspensions of oil in water, have also been used as vehicles for corticosteroids. The compositions of creams vary and are far less greasy than ointments but do not provide the same degree of hydration to the skin, and therefore may not have as high penetration as ointments. Lotions, which are suspensions of oil in water and are similar to creams, are vehicles which include agents to help solubilize the corticosteroids. Solutions have been used as vehicles and are water based with propylene glycol. Gels are solid components at room temperature but melt on the skin. Lotions, gels and solutions have less penetration than ointments.

Many vehicles for corticosteroids include propylene glycol for dissolving the corticosteroid in the vehicle. In general, compositions that contain higher amounts of propylene glycol tend to be more potent.

Due to the effect of the vehicle on potency, different formulations containing the same amount of the same corticosteroid in different vehicles are often in different potency classes. For example, commercially available preparations of 0.05% betamethasone dipropionate are classified as having Class 1, Class 2 or Class 3 potency, depending on their vehicles.

Fluocinonide is a synthetic corticosteroid with clinically proven anti-inflammatory and anti-pruritic therapeutic efficacy. Fluocinonide is a corticosteroid which is the 21-acetate ester of fluocinolone acetonide with the chemical name pregna-1,4-diene-3,20-dione,21-(acetyloxy)-6,9-difluoro-11-hydroxy-16, 17-[(1-methylethylidene)bis(oxy)]-,(6α., 11β,16α)-. It is one member of a large class of gluococorticoids which are used topically for the treatment of certain hyperproliferative and/or inflammatory dermatoses, including atopic dermatitis and psoriasis.

Although the anti-inflammatory mechanism of action of topical steroids is still unclear, corticosteroids are thought to act by the induction of phospholipase A₂ inhibitory proteins, collectively called “lipocortins.” It is postulated that lipocortins control the biosynthesis of potent mediators of inflammation, such as prostaglandins and leukotrienes, by inhibiting the release of arachidonic acid, which in turn is released from the membrane phospholipids by the enzyme phospholipase A₂. The interdependent feedback mechanism between the hypothalamus (responsible for secretion of corticotrophin-releasing factor), the pituitary gland (responsible for secretion of adrenocorticotropic hormone), and the adrenal cortex (which secretes cortisol) is termed “the hypothalamic-pituitary-adrenal (HPA)-axis.” The HPA-axis may be suppressed by topical corticosteroids. The extent of adrenal suppression is generally related to the potency of the topical corticosteroids, the frequency of application, the patient's body surface area (BSA), and the skin's ability to act as a barrier.

Due to the unwanted effects associated with HPA-axis suppression, steroids are generally not recommended for administration to pediatric patients.

### BACKGROUND OF THE INVENTION

Atopic dermatitis is a chronic inflammatory pruritic skin disease which occurs most frequently in pediatric patients and follows a relapsing course. It is often associated with elevated serum immunoglobulin (IgE) levels and a personal or family history of allergies, allergic rhinitis and asthma. Atopic dermatitis is responsive to treatment with corticosteroids. Topical corticosteroids may be absorbed systematically and may suppress the hypothalamus-pituitary-adrenal (HPA) axis. The risk of HPA axis suppression is one of the main safety issues to be considered when prescribing topical corticosteroids. The risk of corticosteroid-induced HPA axis suppression is presumed to be greater in the pediatric population than in adults. As a result of this increasing risk with increasing potency, corticosteroids of greater potency are generally not recommended for use in pediatric patients (i.e., patients less than 18 years in age).

Some corticosteroids have been approved for use in pediatric patients under restrictions as to the age group to be treated and/or the length of the treatment. For example, Cutivate Cream (0.05%), a medium potency corticosteroid, has been approved for children 3 months of age or older. The rate of HPA-axis suppression in children 3 months to 5 years old treated with Cutivate Cream has been reported to be 5%. Elocon Cream (0.1%), another medium potency corticosteroid, has not been approved in patients younger than two years old due to a 16% rate of HPA-axis suppression in children from 6 months to less than two years old. Similarly, Elocon Ointment (0.1%), a medium potency corticosteroid, has not been approved in patients younger than two years due to a 27% rate of HPA-axis suppression in children in the age group from 6 months to less than 2 years old. Clobevate Gel (0.05%), which is considered to be a super-high potency corticosteroid, is not recommended for children under 12 years of age. Clobex Lotion (0.05%), another super-high potency corticosteroid, is only indicated for patients 18 years of age or older because of a 64% rate of HPA-axis suppression in children age 12 through less than 18. Temovate (0.05%) Cream, Gel, Emollient, and Ointment, all considered to be super-high potency corticosteroids, are not recommended for use in patients under 12 years in age. Diprolene AF Cream (0.05%), a high potency steroid, is not indicated for children less than 13 years in age. HPA-axis suppression rates of 50% (for children 3 months to less than two years in age); 38% (for children 2 years to less than 6 years in age); 32% (for children 6 years to less than 9 years in age); and 17% (for children 9 years to less than 12 years in age) were reported for Diprolene AF Cream.

As seen from the data listed above, while some corticosteroids have been approved for limited use in pediatric patients, the rates of HPA-axis suppression are still rather high, resulting in age limitations in the pediatric patients to be treated. Furthermore, no super-high potency corticosteroids have been approved for use in very young pediatric patients. Thus, a need exists for a method of treating pediatric patients suffering from corticosteroid-responsive dermatoses with a super-high potency corticosteroid that results in little or no HPA-axis suppression in all age groups.

### SUMMARY OF THE INVENTION

The present invention comprises a method of treating corticosteroid-responsive dermatoses in pediatric patients with topical fluocinonide, with little or no suppression of the HPA-axis. Pediatric patients may be more susceptible to systemic toxicity from the use of topical steroids because of their larger skin surface to body mass ratios. Pediatric patients are at a greater risk than adult patients of adrenal insufficiency during or after withdrawal of treatment. The use of topical steroids in pediatric patients has resulted in adverse effects including striae, HPA-axis suppression, Cushing's syndrome, linear growth retardation, delayed weight gain, and intracranial hypertension.

According to the present invention, a class I steroid, such as fluocinonide, may be safely administered topically to pediatric patients once or twice daily, with little or no suppression of the HPA-axis. Fluocinonide compositions may be formulated at various potencies depending on the vehicle used and the amount of fluocinonide added to the composition. Fluocinonide (0.1% cream), formulated as a class I steroid, administered once daily does not result in suppression of the HPA-axis in patients 3 months to <18 years of age. Fluocinonide (0.1% cream), formulated as a Class I steroid, administered twice daily may be responsible for a low incidence of suppression of the HPA-axis. Fluocinonide 0.1%, formulated as a Class I steroid, administered once or twice daily is safe, as measured by serum cortisol levels before and after cosyntropin stimulation, evaluation of adverse events, signs and symptoms of skin disease, and vital signs measurements in atopic dermatitis patients 3 months to <18 years of age.

### DETAILED DESCRIPTION OF THE INVENTION

The present invention is directed to a method of treating inflammatory and pruritic skin disorders in pediatric patients with topical fluocinonide. The skin disorders contemplated by the present invention include, but are not limited to, atopic dermatitis and psoriasis. It has been shown that topical application of fluocinonide, once or twice daily, in pediatric patients (i.e., persons less than eighteen years of age) results in little or no suppression of the HPA axis. This result is surprising and unexpected in light of the prior art methods, all of which involve treatment of pediatric patients with compositions having higher rates of HPA-axis suppression than the compositions used in the instant invention. Further, currently, no method exists for treating very young pediatric patients with super-high potency corticosteroids.

A study was performed in pediatric patients with clinically diagnosed atopic dermatitis which demonstrated that fluocinonide administered topically once or twice daily does not result in any significant level of HPA-axis suppression.

Subjects/Methods

At the baseline visit, physical examinations were performed, including measurements of height, weight, and vital signs, and female subjects of childbearing potential underwent a urine pregnancy test (UPT). Patients with any significant disease of the hepatic, renal, endocrine, musculoskeletal, or nervous system were excluded. At Screening/Baseline, each qualified patient was assessed for a normally functioning HPA axis. Male or female patients with clinically diagnosed atopic dermatitis, involving ≧20% of the total body surface area (BSA), were randomized to receive either qd (once a day) or bid (twice a day) treatment in 4 sequential cohorts. The “rule of nines” method was used in calculating affected BSA.

Enrollment in the 4 cohorts was as follows: Cohort 1-33 patients, 12 to <18 years of age; Cohort 2-32 patients, 6 to <12 years of age; Cohort 3-30 patients, 2 to <6 yea Cohort 4-31 infants, 3 months to <2 years of age.

Enrollment into each cohort began only after evaluation of the preceding cohort in order to minimize the risk of systemic toxicity to the study subjects. Four observations of HPA-axis suppression in a single treatment group per cohort were considered as evidence of a clinically significant risk.

Patients who met the inclusion/exclusion criteria were randomized to receive topical fluocinonide either qd or bid. Patients/guardians were instructed to apply a thin layer of the study product to all treatable areas once or twice daily for 14 days. If the areas to be treated were hairy, patients/guardians were instructed to part the hair to allow direct contact between the study product and the lesion. Patients/guardians were also instructed not to apply study product to lesions of the face, groin, perianal area, and axillae. At the end of Weeks 1, 2, and 4, patients returned to the study site in order for the investigator to perform designated evaluations, review treatment compliance and concomitant therapy, and to collect information regarding adverse events.

The potential of fluocinonide to suppress the HPA-axis was assessed by determining the rate of incidence of suppression, when suppression was defined as a serum cortisol level ≦18 μg/dL, 30 minutes after intravenous cosyntropin stimulation. For a subject to be evaluable for HPA-axis suppression, the subject must have received at least 2 full weeks of fluocinonide treatment prior to the cosyntropin challenge. Pre-and post-cosyntropin-stimulation blood samples were obtained between 7:30 and 8:30 AM to account for diurnal variation in cortisol levels, at the Baseline and Week 2 visits, as directed in the Cortrosyn® package insert. Cortrosyn® is a drug that stimulates the adrenal gland. In instances of HPA-axis suppression, the blood cortisol levels do not rise above 18 μg/dL after administration of Cortrosyn®. The dose and administration of cosyntropin followed the labeling of the product. Specifically, the dose for subjects 3 to 17 years of age was 0.25 mg, and the dose for subjects 3 months to 2 years old was 0.125 mg. At the end of the 14-day treatment period, any subject with a post-stimulation cortisol level ≦18 μg/dL was re-tested at Week 4 and once every 4 weeks thereafter until the post-stimulation levels were within normal limits.

Specific skin safety evaluations were performed at each study visit with regard to all treated lesions by noting the presence or absence of the following 8 signs and symptoms of skin atrophy: telangiectasia, transparency, loss of elasticity, loss of normal skin markings, thinning, striae, pigmentation changes, and bruising.

Adverse events were coded using MedDRA terminology (a standard dictionary of terms used to clarify adverse events) and were described according to their intensity, duration and relationship to the study product.

Efficacy was assessed by observing the change in the severity of atopic dermatitis from Baseline to Week 2 and Week 4. A disease exacerbation requiring more aggressive treatment than previously used was considered an adverse event.

Mean post-stimulation cortisol levels measured at Baseline and at Week 2 were compared using a paired t test.

Results

In Cohort 1 (patients aged 12 to <18 years), a total of 33 patients were randomized into the study: 16 were treated with fluocinonide qd, and 17 with fluocinonide bid. Thirty patients in Cohort 1 completed the study and 3 patients discontinued the study. One patient in the qd group discontinued due to an adverse event (moderate urticaria) and 2 patients in the bid group discontinued for other reasons (cortisol suppression noted at Screening/Baseline in one patient and at Baseline in the other patient). In Cohort 2 (patients aged 6 to <12 years), a total of 32 patients were randomized into the study: 16 were treated with fluocinonide qd and 16 with fluocinonide bid. Thirty-one patients in Cohort 2 completed the study. One patient in the qd group, who had entered in violation of enrollment criteria (cortisol suppression noted at Screening), was discontinued from the study prior to Day 7. In Cohort 3 (patients aged 2 to <6 years), a total of 30 patients were randomized into the study: 15 were treated with fluocinonide qd and 15 with fluocinonide bid. All 30 patients in Cohort 3 completed the study. In Cohort 4 (patients aged 3 months to <2 years), a total of 31 patients were randomized into the study: 16 were treated with fluocinonide qd and 15 with fluocinonide bid. Thirty patients in Cohort 4 completed the study. One patient in the fluocinonide qd treatment group was treated with study medication for only 7 days.

At Baseline, the mean percentage of BSA affected by atopic dermatitis (BSA involvement) ranged from approximately 34% in the Cohort 1 fluocinonide bid group to 43% in the Cohort 4 fluocinonide qd group.

The demographic characteristics are summarized in Table 1 below.

TABLE 1
Demographic summary of all patients

| | Cohort: | | | | | | | |
|---|---|---|---|---|---|---|---|---|
| | 1 | | 2 | | 3 | | 4 | |
| | Cohort age: | | | | | | | |
| | | | | | | | 3 months to <2 | |
| | (12-<18) | | (6-<12) | | (2-<6) | | year | |
| | Fluocinonide regimen: | | | | | | | |
| | qd | bid | qd | bid | qd | bid | qd | bid |
| Patients Studied, N | 16 | 17 | 16 | 16 | 15 | 15 | 16 | 15 |
| Age (year) | | | | | | | | |
| Mean ± SD | 14.6 ± 1.5 | 14.4 ± 1.7 | 8.6 ± 1.9 | 8.9 ± 1.6 | 3.7 ± 1.2 | 3.3 ± 1.0 | 1.3 ± 0.4 | 1.2 ± 0.5 |
| Min-Max | 12.1-16.9 | 12.0-17.8 | 6.1-11.5 | 6.3-11.3 | 2.1-5.9 | 2.2-4.8 | 0.5-1.9 | 0.7-1.9 |
| Gender - n (%) | | | | | | | | |
| Male | 7 (43.6) | 6 (35.3) | 8 (50.0) | 8 (50.0) | 6 (40.0) | 10 (66.7) | 12 (75.0) | 10 (66.7) |
| Female | 9 (56.3) | 11 (64.7) | 8 (50.0) | 8 (50.0) | 9 (60.0) | 5 (33.3) | 4 (25.0) | 5 (33.3) |
| Race - n (%) | | | | | | | | |
| Caucasian | 10 (62.5) | 13 (76.5) | 12 (75.0) | 8 (50.0) | 9 (60.0) | 11 (73.3) | 15 (93.8) | 10 (66.7) |
| Black | 4 (25.0) | 3 (17.6) | 1 (6.3) | 5 (31.3) | 5 (33.3) | 1 (6.7) | 0 | 3 (20.0) |
| Other* | 2 (12.5) | 1 (5.9) | 3 (18.8) | 3 (18.9) | 1 (6.7) | 3 (20.0) | 1 (6.3) | 2 (13.3) |
| Duration of disease | | | | | | | | |
| (years) | | | | | | | | |
| Mean ± SD | 12.1 ± 4.5 | 11.8 ± 4.8 | 7.3 ± 2.9 | 7.0 ± 2.4 | 3.0 ± 1.6 | 3.0 ± 1.0 | 1.1 ± 0.5 | 1.2 ± 0.5 |
| Min-Max | 0.6-16.6 | 0.3-17.8 | 1.6-11.5 | 0.6-10.7 | 0.5-5.5 | 1.3-4.8 | 0.2-1.9 | 0.5-1.9 |
| BSA involvement | | | | | | | | |
| (%) | | | | | | | | |
| Mean ± SD | 36.1 ± 19.7 | 34.0 ± 14.9 | 40.7 ± 18.1 | 40.6 ± 22.7 | 34.9 ± 18.5 | 34.2 ± 12.0 | 43.1 ± 18.5 | 38.2 ± 18.1 |
| Min-Max | 20.0-92.0 | 20.0-75.0 | 20.0-90.0 | 20.0-95.0 | 20.0-90.0 | 20.0-57.0 | 23.0-80.0 | 20.0-80.0 |

SD—Standard Deviation;
BSA—body surface area
*Other indicates Asian, Hispanic, or Native American

HPA-axis Suppression

Mean post-stimulation cortisol levels ranged from 23.8 μg/dL to 30.3 μg/dL across all cohort and treatment groups, at Screening and at Week 2. Post-stimulation levels were statistically significantly lower at Week 2 compared to Screening for the Cohort 2 qd regimen (28.0 ug/dL vs. 24.7 ug/dL, P=0.015) but remained within normal limits. There were no other statistically significant changes in post-stimulation cortisol levels in any cohort or regimen.

One patient of 15 (6.7%) in the Cohort 1 fluocinonide bid group met the criterion for HPA-axis suppression, with a post-stimulation serum cortisol level at Week 2 of 17.6 μg/dL (Screening value of 20.4 μg/mL). At the Week 4 visit (2 weeks post-treatment), the value was normal, at 18.9 μg/dL.

Two patients of 16 (12.5%) in the Cohort 2 fluocinonide bid treatment group met the criterion for HPA-axis suppression. The post-stimulation serum cortisol level of the first patient at Week 2 was 17.2 μg/dL (Screening value of 32.1 μg/dL). At the Week 4 visit (2 weeks post-treatment), the value for this patient was normal, at 28.7 μg/dL. The post-stimulation serum cortisol level for the second patient at Week 2 was 16.7 μg/dL (Screening value of 20.5 μg/dL). At an unscheduled visit 8 days after the end of treatment, the value for this patient was normal, at 22.4 μg/dL.

One patient of 15 (6.7%) in the Cohort 3 fluocinonide bid treatment group met the criterion for HPA-axis suppression at the Week 2 visit. The post-stimulation serum cortisol level for the patient at Week 2 was 9.1 μg/dL, compared to the pre-stimulation level of 28.0 μg/dL. Because the pre-stimulation cortisol level was higher than the post-stimulation level, it was suspected that the pre-stimulation and post-stimulation samples were reversed and that the HPA-axis response to cosyntrophin stimulation in this subject was normal. Post-stimulation values at Screening and Week 4 were 40.0 μg/dL and 30.3 μg/dL, respectively. As the pre-stimulation value was >18 μg/dL, this subject cannot be said to have suppression of the HPA-axis. None of the subjects in Cohort 4 experienced HPA-axis suppression.

This study provides evidence that younger patients do not have an increased risk of HPA-axis suppression associated with treatment with fluocinonide formulated as a Class I steroid. In both the youngest and the oldest cohorts, the mean response to intravenous Cortrosyn challenge stayed the same after treatment with fluocinonide qd, whereas in the fluocinonide bid groups of Cohorts 1 and 4, and in both treatment groups of Cohorts 2 and 3, the mean response to Cortrosyn decreased slightly. HPA-axis suppression was observed in 1/15 (6.7%) patients in the fluocinonide bid group in Cohort 1, and in 2/16 (12.5%) patients in the fluocinonide bid group in Cohort 2. The difference in the incidence of HPA-axis suppression in the two treatment groups was not statistically significant. Current topical corticosteroid labeling suggests that pediatric patients may be more susceptible to HPA-axis suppression due to the larger skin surface-to-body mass ratio. However, according to the method of the present invention, there is no evidence of greater susceptibility in younger patients when treated with fluocinonide, even when treating large portions of diseased body surfaces. While the fluocinonide tested is rated as a Class I steroid, similar results are expected for all degrees of potency of fluocinonide since Class I steroids are the strongest and little or no HPA-axis suppression was demonstrated.

These data are summarized in Table 2 below.

TABLE 2
Summary of cortisol levels in response to cosyntropin stimulation response-
evaluable population

| | Cohort: | | | | | | | |
|---|---|---|---|---|---|---|---|---|
| | 1 | | 2 | | 3 | | 4 | |
| | Fluocinonide regimen: | | | | | | | |
| | qd | bid | qd | bid | qd | bid | qd | bid |
| Evaluable Subjects | 15 | 15 | 15 | 16 | 15 | 15 | 15 | 15 |
| (N): | | | | | | | | |
| Screening | | | | | | | | |
| Pre-stimulation | | | | | | | | |
| level | | | | | | | | |
| Mean ± SD | 17.2 ± 6.6 | 15.7 ± 10.3 | 14.0 ± 5.0 | 12.5 ± 4.5 | 11.6 ± 3.6 | 14.4 ± 5.6 | 11.8 ± 4.3 | 11.3 ± 4.9 |
| Range (μg/dL) | 2.0-26.1 | 0.9-42.7 | 6.6-25.1 | 6.4 ± 22.2 | 7.3-19.8 | 6.1-23.4 | 4.7-19.6 | 6.0-20.8 |
| Post-stimulation | | | | | | | | |
| level | | | | | | | | |
| Mean ± SD | 26.4 ± 3.7 | 26.8 ± 8.0 | 28.0 ± 4.3 | 24.9 ± 3.9 | 29.0 ± 3.8 | 29.9 ± 6.1 | 28.3 ± 5.1 | 29.2 ± 6.1 |
| Range (μg/dL) | 18.2-31.3 | 18.3-50.4 | 20.9-36.2 | 19.4-32.1 | 22.8-37.0 | 20.1-40.0 | 20.7-36.2 | 20.5-38.8 |
| Increase in levels | | | | | | | | |
| Mean ± SD | 9.3 ± 5.2 | 11.1 ± 3.7 | 14.0 ± 6.1 | 12.3 ± 3.7 | 17.4 ± 3.1 | 15.6 ± 7.0 | 16.5 ± 4.7 | 17.9 ± 4.7 |
| Range (μg/dL) | −1.2-22.1 | 4.3-17.4 | 3.0-22.6 | 3.8-17.8 | 11.2-23.0 | 1.8-30.0 | 6.1-23.0 | 11.4-25.0 |
| End of Week 2 | | | | | | | | |
| Pre-stimulation | | | | | | | | |
| level | | | | | | | | |
| Mean ± SD | 17.6 ± 7.6 | 17.2 ± 8.3 | 13.7 ± 5.6 | 12.3 ± 4.9 | 15.4 ± 7.0 | 15.0 ± 5.9 | 10.4 ± 3.2 | 13.0 ± 4.6 |
| Range (μg/dL) | 1.2-32.9 | 7.1-34.2 | 6.9-29.3 | 6.3-24.2 | 8.6-36.4 | 4.6-28.0 | 4.6-14.4 | 5.8-22.6 |
| Post-stimulation | | | | | | | | |
| level | | | | | | | | |
| Mean ± SD | 26.9 ± 4.9 | 25.9 ± 6.8 | 24.7 ± 3.0* | 23.8 ± 5.5 | 28.1 ± 4.5 | 25.9 ± 6.3 | 30.3 ± 6.6 | 29.5 ± 7.7 |
| Range | 21.0-39.9 | 17.6-40.6 | 20.5-31.4 | 16.7-37.7 | 22.1-39.5 | 9.1-33.7 | 19.1-38.8 | 20.0-49.5 |
| Increase in levels | | | | | | | | |
| Mean ± SD | 9.3 ± 5.1 | 8.6 ± 4.7 | 11.0 ± 4.8 | 11.5 ± 5.2 | 12.7 ± 4.9 | 11.0 ± 10.4 | 19.9 ± 7.1 | 16.5 ± 6.6 |
| Range (μg/dL) | 3.5-22.2 | 1.2-17.0 | −2.3-17.8 | −3.5-20.3 | 3.1-22.0 | −19-29.1 | 6.4-29.9 | 7.0-29.7 |
| HPA-Axis | 0 (0.0) | 1 (6.7) | 0 (0.0) | 2 (12.5) | 0 (0) | 0 (0) | 0 (0.0) | 0 (0.0) |
| suppression (N) | | | | | | | | |
| Not suppressed | 15 (100) | 14 (93.3) | 15 (100) | 14 (87.5) | 15 (100) | 15 (100) | 15 (100) | 15 (100) |

SD—Standard Deviation
*Statistically significant changes from screening (P = 0.015), post-stimulation levels were within normal limits.

Skin Safety Evaluations

In 1 or 2 patients in each of the 4 cohorts and treatment groups, one or more signs of skin atrophy such as loss of elasticity, loss of normal skin markings, skin thinning, striae, and pigmentation changes were observed post-Baseline, but were considered unrelated to the treatment with fluocinonide. Overall, there were 5/62 (8.1%) patients in the fluocinonide qd treatment group and also 5/62 (8.1%) patients in the bid treatment group who showed new signs of skin atrophy at Week 2 that were not present at Baseline. Thus, there were no age-related or dose-related differences in the incidence of these effects.

A summary of the skin safety assessments is contained in Table 3 below.

TABLE 3
Summary of skin safety assessments

| | Cohort: | | | | | | | |
|---|---|---|---|---|---|---|---|---|
| | 1 | | 2 | | 3 | | 4 | |
| | Cohort age: | | | | | | | |
| | (12-<18) | | (6-<12) | | (2-<6) | | 3 months to <2 year | |
| | Fluocinonide regimen: | | | | | | | |
| | qd | bid | qd | bid | qd | bid | qd | bid |
| Patients | 15 | 16 | 16 | 16 | 15 | 15 | 16 | 15 |
| presenting data | | | | | | | | |
| at Week 2, N | | | | | | | | |
| Number (%) of | 2 (13.3) | 2 (12.5) | 0 | 1 (6.3) | 1 (6.7) | 0 | 2 (12.5) | 2 (13.3) |
| patients who | | | | | | | | |
| showed new | | | | | | | | |
| signs at | | | | | | | | |
| Week 2 not | | | | | | | | |
| present at | | | | | | | | |
| Baseline | | | | | | | | |
| New signs | striae, | loss of | | change in | change in | | skin | change in |
| noted | bruising | elasticity, | | pigmentation | pigmentation | | transparency, | pigmentation |
| | | loss of | | | | | skin thinning, | bruising |
| | | skin | | | | | change in | |
| | | markings | | | | | pigmentation | |

Adverse Events

Forty-five of 126 patients (35.7%) treated in this study reported at least one treatment-emergent adverse event (TEAE). No age-related trends were evident with regard to the incidence of treatment-related adverse events.

One patient in the Cohort 1 fluocinonide qd treatment group was withdrawn from the study due to an adverse event, moderate urticaria. No patients were withdrawn due to adverse events in Cohorts 2, 3 or 4. No deaths or other serious adverse events occurred during the study.

A summary of adverse events is contained in Table 4 below.

TABLE 4
Treatment-emergent adverse events which occurred in 2 or more patients in
any cohort/regimen, Number (%)

| | Cohort: | | | | | | | |
|---|---|---|---|---|---|---|---|---|
| | 1 | | 2 | | 3 | | 4 | |
| | Fluocinonide: | | | | | | | |
| | qd | bid | qd | bid | qd | bid | qd | bid |
| Patients studied | | | | | | | | |
| Total no. of patients | 16 (100) | 17 (100) | 16 (100) | 16 (100) | 15 (100) | 15 (100) | 16 (100) | 15 (100) |
| Total no. of patients with | 6 (37.5) | 9 (52.9) | 4 (25.0) | 6 (37.5) | 3 (20) | 2 (13.3) | 8 (50.0) | 7 (46.7) |
| AEs | | | | | | | | |
| Headache | 1 (6.3) | 3 (17.6) | 0 (0) | 0 (0) | 0 (0) | 0 (0) | 0 (0) | 0 (0) |
| Burning Sensation NOS | 0 (0) | 2 (11.8) | 0 (0) | 0 (0) | 0 (0) | 0 (0) | 0 (0) | 0 (0) |
| Folliculitis | 2 (12.5) | 1 (5.9) | 1 (6.3) | 0 (0) | 0 (0) | 0 (0) | 0 (0) | 0 (0) |
| Pain NOS | 0 (0) | 2 (11.8) | 1 (6.3) | 0 (0) | 0 (0) | 0 (0) | 0 (0) | 0 (0) |
| Upper respiratory tract | 0 (0) | 0 (0) | 0 (0) | 1 (6.3) | 0 (0) | 0 (0) | 2 (12.5) | 1 (6.7) |
| infection | | | | | | | | |
| Gastroenteritis NOS | 0 (0) | 0 (0) | 0 (0) | 0 (0) | 0 (0) | 0 (0) | 2 (12.5) | 0 (0) |
| Pyrexia | 0 (0) | 0 (0) | 0 (0) | 0 (0) | 0 (0) | 0 (0) | 2 (12.5) | 2 (13.3) |
| Dermatitis diaper | 0 (0) | 0 (0) | 0 (0) | 0 (0) | 0 (0) | 0 (0) | 2 (12.5) | 0 (0) |

qd—quaque die (once daily);
bid—bis in die (twice daily);
AE—adverse events;
NOS—not otherwise specified

Efficacy

In both Cohort 1 and Cohort 2, all of the patients in the fluocinonide qd group and the majority (>90%) of the patients in the fluocinonide bid group were rated by the investigator as showing an improvement in their disease status or as clear or almost clear of disease at the end of the treatment period compared to Baseline. In Cohorts 3 and 4, more than 90% of both the qd and bid groups were rated by the investigator as showing an improvement in their disease status or as clear or almost clear of disease at the end of the treatment period. At the 2-week follow-up visit, >90% of the patients in each dosage group in Cohort 4, >80% of patients in each dosage group in Cohort 1 and Cohort 3 and >70% of patients in each dosage group in Cohort 2 continued to show an improvement of clear or almost clear compared to Baseline.

The sole efficacy variable in the study was an investigator rating of skin disease severity performed at Screening, at end of treatment, and at a follow-up visit 4 weeks after the start of study treatment. Fluocinonide administered either qd or bid appears to be effective against atopic dermatitis in children aged 3 months to 17 years. In each of the 4 cohorts of this study, more than 90% of the patients in both the fluocinonide qd and bid group were rated by the investigator as showing an improvement in their disease status or as clear or almost clear of disease at the end of the treatment period. The qd regimen appeared to be as effective as the bid regimen across all age groups.

### Embodiments

In one embodiment of the invention, the fluocinonide may be delivered in a vehicle comprising at least two penetration enhancers, including diisopropyl adipate, dimethyl isosorbide, propylene glycol, 1,2,6-hexanetriol, and benzyl alcohol.

According to another embodiment of the present invention, fluocinonide is combined with two or more penetration enhancers (preferably propylene glycol and at least one other penetration enhancer), and one or more solvents and emulsifiers for the corticosteroid and optionally penetration enhancers, wherein the penetration enhancers are present in ratio to the total of the penetration enhancers, and solvents and emulsifiers of at least about 0.70, preferably at least 0.80 and most preferably 0.90 or 0.95. Optionally, one or more inactive ingredients may also be combined with fluocinonide.

In order to determine the ratio, the following formula is used: (a:(a+b)) where “a” is the penetration enhancers and “a+b” is the sum of penetration enhancers, and solvents and emulsifiers. In the ratio of (a): (a+b), penetration enhancers include at least two of: propylene glycol, diusopropyl adipate, dimethyl isosorbide, 1,2,6 hexanetriol, and benzyl alcohol (collectively referred to as “a”). The solvents and emulsifiers for the corticosteroid include one or more of dehydrated alcohol, alcohol (95% v/v) USP, 3-Cyclohexene-1-Methanol, .varies.4-Dimethyl-a-(4-Methyl-3-Pentenyl)-, Steareth-2, Steareth-21, citric acid, CPE-215, diisopropanolamine (1:9), DIPA/PG (1:9), ethoxydiglycol, Potassium hydroxide (10%), PEG-40 Stearate, PEG-7000, Polysorbate 60, potassium hydroxide (1%), propylene carbonate USP, propylethylene glycol 4, oleyl alcohol, sodium lauryl sulfate, sorbitan monostearate, sorbitan stearate, and 1,2,3-Propanetriol Ester (collectively referred to as “b”).

The compositions optionally comprise non-solvent/emulsifier ingredients, such as Glyceryl Stearate (and) PEG-100 Stearate, carbopol 980, cyclomethicone NF, glyceryl monostearate, hydroxyethyl cellulose, hydroxypropyl cellulose, isopropyl myristate, methyl paraben NF, mineral oil, oleic acid NF, PEG-100 Stearate, petrolatum, propyl paraben NF, purified water, stearyl alcohol, white petrolatum, and white wax.

One embodiment of a fluocinonide composition that can be used in pediatric patients is detailed in the chart below.

| Component | % w/w | % w/w |
|---|---|---|
| Fluocinonide Micronized, | 0.1 | 0.1 |
| USP | | |
| Propylene Glycol, USP | 70.0 | 74.9 |
| Dimethyl isosorbide | 15.0 | |
| Diisopropyl Adipate | | 3.0 |
| Isopropyl Myristate, NF | | 5.0 |
| 1,2,6 Trihydroxyhexane | | 2.5 |
| Carbopol 980 | 1.2 | 1.0 |
| Diisopropanolamine | 1.2 | 1.0 |
| 85%: propylene glycol (1:9) | | |
| Citric Acid, USP | 0.01 | 0.01 |
| Purified Water, USP | 2.49 | 2.49 |
| Glyceryl monostearate | 2.5 | 2.5 |
| Glyceryl monostearate & PEG | 7.5 | 7.5 |
| stearate | | |

Another embodiment of a composition that can be used in accordance with the present invention is detailed in the chart below.

| Component | % w/w | % w/w |
|---|---|---|
| Fluocinonide Micronized, | 0.1 | 0.1 |
| USP | | |
| Propylene Glycol, USP | 66.8 | 69.9 |
| Dimethyl isosorbide | 5.0 | |
| Diisopropyl Adipate | | 2.0 |
| Isopropyl Myristate, NF | 5.0 | 5.0 |
| Carbopol 980 | 0.5 | 0.5 |
| Diisopropanolamine | 0.5 | 0.5 |
| 85%: propylene glycol (1:9) | | |
| White Petrolatum, USP | 5.0 | 5.0 |
| Glyceryl monostearate | 6.0 | 6.0 |
| PEG 100 stearate | 6.0 | 6.0 |
| Stearyl alcohol, NF | 5.0 | 5.0 |
| Sodium Lauryl Sulfate, NF | 0.1 | |

A further embodiment of a composition that can be used in accordance with the present invention is detailed in the chart below.

| Component | % w/w |
|---|---|
| Propylene glycol, USP | 71.08 |
| Dimethyl isosorbide | 15.00 |
| Arlacel 165 (Glyceryl | 7.50 |
| monostearate and PEG | |
| stearate) | |
| Glyceryl monostearate, NF | 2.50 |
| Purified water, USP | 2.49 |
| Carbopol 980 | 1.20 |
| Diisopropanolamine 85% | 0.12 |
| Fluocinonide micronized, USP | 0.10 |
| Citric acid, USP | 0.01 |

It is to be understood that while the invention has been described in conjunction with the detailed description thereof, that the foregoing description is intended to illustrate and not limit the scope of the invention, which is defined by the scope of the appended claims. Other aspects, advantages, and modifications are evident from a review of the following claims.

What is claimed is:

1. A method of treating pediatric patients suffering from a corticosteroid-responsive inflammatory or pruritic skin disorder comprising topically applying fluocinonide to an affected area wherein said application does not result in clinically significant HPA-axis suppression.

2. The method of claim 1 wherein the inflammatory or pruritic skin disorder is atopic dermatitis.

3. The method of claim 1 wherein the inflammatory or pruritic skin disorder is psoriasis.

4. The method of claim 1 wherein the fluocinonide is applied one time per day.

5. The method of claim 1 wherein the fluocinonide is applied two times per day.

6. The method of claim 1 wherein the fluocinonide is applied at a concentration of 0.1% by weight.

7. A method of treating pediatric patients suffering from a corticosteroid-responsive inflammatory or pruritic skin disorder comprising topically applying a composition comprising a corticosteroid; two or more penetration enhancers selected from the group consisting of diisopropyl adipate, dimethyl isosorbide, propylene glycol, 1,2,6-hexapetriol, and benzyl alcohol; and one or more of the group consisting of solvents and emulsifiers, wherein the penetration enhancers are present in a ratio to a total of the penetration enhancers, and solvents and emulsifiers of at least about 0.90, to an affected area wherein said application does not result in clinically significant HPA-axis suppression.

8. The method of claim 7 wherein the corticosteroid is fluocinonide.

9. The method of claim 7 wherein the inflammatory or pruritic skin disorder is atopic dermatitis.

10. The method of claim 7 wherein the inflammatory or pruritic skin disorder is psoriasis.

11. The method of claim 7 wherein the composition is applied one time per day.

12. The method of claim 7 wherein the composition is applied two times per day.

13. The method of claim 7 wherein the corticosteroid is applied at a concentration of 0.1% by weight.

14. A method of treating pediatric patients suffering from a corticosteroid-responsive inflammatory or pruritic skin disorder comprising topically applying a composition comprising 71.08% propylene glycol USP, 15.00% dimethyl isosorbide, 7.50% glyceryl monostearate and PEG stearate, 2.50% glyceryl monostearate NF, 2.49% purified water, 1.20% carbopol 980, 0.12% diisopropanolamine 85%, 0.10% fluocinonide (micronized) USP, and 0.01% citric acid USP, to an affected area wherein said application does not result in clinically significant HPA-axis suppression.

15. The method of claim 14 wherein the inflammatory or pruritic skin disorder is atopic dermatitis.

16. The method of claim 14 wherein the inflammatory or pruritic skin disorder is psoriasis.

17. The method of claim 14 wherein the composition is applied one time per day.

18. The method of claim 14 wherein the composition is applied two times per day.
